(19) United States
(12) Patent Application Publication
(10) Pub. No.: US 2024/0423819 A1
(43) Pub. Date: Dec. 26, 2024

(54) MEDICAL DEVICES AND COATING METHOD

(71) Applicant: SUZHOU LAVAMED CO., LTD., Suzhou, Jiangsu (CN)

(72) Inventors: Xin Hua, Suzhou, Jiangsu (CN); Liam Farrissey, Oranmore, Co. Galway (IE); Brendan Cunniffe, Oranmore, Co. Galway (IE); Quan Shen, Suzhou, Jiangsu (CN)

(21) Appl. No.: 18/685,168

(22) PCT Filed: Aug. 22, 2022
(86) PCT No.: PCT/CN2022/114039

§ 371 Date: Feb. 20, 2024

(30) Foreign Application Priority Data
Aug. 20, 2021 (WO) PCT/CN2021/113777

(51) Int. Cl. A61F 2/90; A61L 33/00; A61L 33/02; D04C 1/06; D06M 10/04; D06M 23/06

(52) U.S. Cl. CPC A61F 2/90; A61L 33/0011; A61L 33/0088; A61L 33/0094; A61L 33/022; D04C 1/06; D06M 10/04; D06M 23/06; A61F 2240/002; A61L 2420/02; A61L 2420/08; D06M 2200/00; D06M 2400/01; D10B 2509/06

(57) ABSTRACT

A medical device is provided, comprising: an expandable tubular member comprising a plurality of elements forming a sidewall, wherein each of the elements crosses one or more other elements and a plurality of pores are formed among the elements in the sidewall; wherein an antithrombogenic material is coated over the tubular member such that there is inherently no webbing of the antithrombogenic material between any two elements.

### FIELD OF THE INVENTION

The present invention relates to a medical device and the invention also relates to a method for the coating of the medical device.

### BACKGROUND OF THE INVENTION

Recently, there is an increasing requirement for antithrombotic properties of medical devices, such as stents for intracranial, coronary and peripheral vessels. So continuous improvements in antithrombotic properties of such medical devices are expected. Below is a detailed explanation of such a condition, with stents for aneurysms as an example.

Walls of the vasculature, particularly arterial walls, may develop areas of pathological dilatation called aneurysms. As is well known, aneurysms have thin, weak walls that are prone to rupturing. Aneurysms can be the result of the vessel wall being weakened by disease, injury, or a congenital abnormality. Aneurysms could be found in different parts of the body, and the most common are abdominal aortic aneurysms and brain or cerebral aneurysms in the neurovasculature. When the weakened wall of an aneurysm ruptures, it can result in death, especially if it is a cerebral aneurysm that ruptures.

Aneurysms are generally treated by excluding the weakened part of the vessel from the arterial circulation. Cerebral aneurysms can be treated by invasive means, such as surgical clipping of the aneurysm, or less invasive endovascular routes, such as filling of the aneurysm sac with embolic devices such as coils or diverting the blood flow away from the sac by placement of a stent across the neck, known as a flow diverter.

Stents include generally tubular prostheses that expand radially or otherwise within a vessel or lumen to provide therapy or support against blockage of the vessel. Stents of various constructions may be utilized, including balloon expandable metal stents, expandable braided metal stents, knitted metal stents, coiled stents, rolled stents, and the like. Stent-grafts are also used, which include a tubular graft material supported by a metallic stent.

Coatings have been applied to medical devices to impart lubricious and/or anti-adhesive properties and serve as depots for bioactive agent release. As medical devices, especially those possessing irregular and/or rough surfaces, may be conducive to thrombus formation, coatings may be applied to these medical devices to reduce the formation of thrombi.

In general, prior coated medical devices, such as stents, have demonstrated various coating imperfections and resultant disadvantages, because in the prior art the coating is formed on the stent with less firmness and with very small thickness. Among these disadvantages, webbing, delamination and uneven layering of coating material pose significant risks. The dislodged coating material can create or contribute to blockages in the blood vessel. They may also interfere with the correct expansion of the device.

### SUMMARY OF THE INVENTION

The present invention discloses a medical device, such as a stent or a braided stent, having a coating where there is inherently no webbing (of the coating material) between any two elements or features (such as filaments) of the stent, particularly between two adjacent elements or features.

Such ‘webbing’ means that a continuous connection of coating exists from one element (or feature) of the device to another and results in bridging across the elements. That is, the elements of the device are not in contact with one another but the coating links one element to the other through a bridge, and such coating constitutes webbing. The webbing can be easy to peel from the elements, and the dislodged webbing can create or contribute to blockages in the blood vessel, into which the medical device is deployed. Further, the webbing has the potential to alter the inherent porosity of the structure of the elements, such as braided structure, thereby affecting the efficacy of the structure in treating the target site.

When the elements of the device are in contact with one another (such as at wire crossover points), it is intended that the coating is continuous from one element to the other only at the crossover points and does not form a bridge of material across the elements that are touching. It is intended that the coating will form a thin continuous coating on the elements themselves but not a bridge of material between two elements. In other words, if the elements of the device are in contact with one another, there is no webbing in any of the filament crossing points.

Even if the two elements are very close, there is also no webbing. In one embodiment, at crossing points between two elements, the distance between the two elements is approaching zero. In one embodiment, the distance between the elements is about 200 microns.

There is also no webbing when the two elements are far enough apart, so there is no webbing at all in any of the pores.

According to one aspect of the invention, there is provided a medical device, comprising:
an expandable tubular member comprising a plurality of elements forming a sidewall, wherein each of the elements crosses one or more other elements and a plurality of pores are formed among the elements in the sidewall;
wherein an antithrombogenic material is coated over the tubular member such that there is inherently no webbing of the antithrombogenic material between any two elements.

According to another aspect of the invention, there is provided a medical device, comprising:
an expandable tubular member having at least a flow diverting portion, the flow diverting portion comprising a plurality of elements forming a sidewall of the tubular member, wherein each of the elements crosses one or more other elements and a plurality of pores are formed among the elements in the sidewall;
wherein the pores are sized to interfere with blood flow to a degree sufficient to thrombose the aneurysm when the tubular member is expanded;
wherein an antithrombogenic material is coated over the flow diverting portion such that there is inherently no webbing of the antithrombogenic material between any two elements.

Preferably, the medical device is for treating an aneurysm.

Preferably, the antithrombogenic material is coated onto the tubular member through a plasma deposition method.

Preferably, the plasma deposition method is a non-thermal plasma deposition method.

Preferably, the medical device is a stent, each of the elements is a filament, and the stents are formed by braiding the filaments.

Preferably, the tubular member is coated with the antithrombogenic material in its entirety.

Preferably, the tubular member is coated with the antithrombogenic material on its inner luminal surface.

Preferably, the antithrombogenic material comprises an antithrombogenic polymer.

According to one aspect of the invention, there is provided a method for coating a medical device, which has a plurality of elements crossing with each other, comprising:
preparing a solution of antithrombogenic material;
nebulizing the solution to form a liquid aerosol;
generating plasma with a plasma source;
introducing the liquid aerosol into the plasma; and
depositing the medical device with a coating of the antithrombogenic material by exposing the medical device to the liquid aerosol and the plasma, so that there is inherently no webbing of the antithrombogenic material between any two elements of the medical device.

Preferably, the medical device is for treating an aneurysm.

Preferably, the plasma operates in a non-thermal manner.

Preferably, the antithrombogenic material is adhered to the surface of the medical device by means of chemical bonding.

Preferably, the medical device is a stent, each of the elements is a filament, and the stent is formed by braiding the filaments.

Preferably, the antithrombogenic material comprises an antithrombogenic polymer.

According to another aspect of the invention, there is provided a medical device, comprising:
an expandable tubular member comprising a plurality of elements forming a sidewall, wherein each of the elements crosses one or more other elements and a plurality of pores are formed among the elements in the sidewall;
wherein an antithrombogenic material is coated over the tubular member, and a coating of the antithrombogenic material formed thereon has a thickness equal to or greater than 10 nm.

According to another aspect of the invention, there is provided a medical device, comprising:
an expandable tubular member having at least a flow diverting portion, the flow diverting portion comprising a plurality of elements forming a sidewall of the tubular member, wherein each of the elements crosses one or more other elements and a plurality of pores are formed among the elements in the sidewall;
wherein the pores are sized to interfere with blood flow to a degree sufficient to thrombose the aneurysm when the tubular member is expanded;
wherein an antithrombogenic material is coated over the flow diverting portion, and a coating of the antithrombogenic material formed thereon has a thickness equal to or greater than 10 nm.

Preferably, the medical device is for treating an aneurysm.

Preferably, wherein the antithrombogenic material is coated onto the tubular member through a plasma deposition method such that there is inherently no webbing of the antithrombogenic material between any two elements.

Preferably, wherein a layer of blue oxide is provided over the tubular member, and the antithrombogenic material is coated over the layer of blue oxide.

Preferably, wherein the coating of the antithrombogenic material on the tubular member has a super-smooth surface.

Preferably, wherein the antithrombogenic material is adhered to the surface of the medical device by means of chemical bonding.

Preferably, wherein a covalent bonding is formed between adjacent particles of the antithrombogenic material and/or a covalent bonding is formed between the antithrombogenic material and the surface of the medical device or the layer of blue oxide during coating.

Preferably, wherein the coating of the antithrombogenic material has a thickness of 10-200 nm.

Preferably, wherein the coating of the antithrombogenic material has a thickness of 40-60 nm.

Preferably, wherein the surface of the coating of the antithrombogenic material has an average roughness of about 0.02-0.2 μm, and particularly 0.1 μm and/or a Mean Roughness depth of about 0.2-2 μm, and particularly 0.5 μm.

Preferably, wherein the medical device is a stent, each of the elements is a filament, and the stents are formed by braiding the filaments.

Preferably, wherein the tubular member is coated with the antithrombogenic material in its entirety.

Preferably, wherein the tubular member is coated with the antithrombogenic material on its inner luminal surface.

Preferably, wherein the antithrombogenic material comprises an antithrombogenic polymer.

The present invention discloses a plasma method that deposits the coating material on the medical device, such as a stent or a braided stent. The plasma method may be all known methods of plasma deposition that could be used to form a coating on the device.

### BRIEF DESCRIPTION OF THE DRAWINGS

FIG. 1 illustrates a tubular, expandable device, with a partial enlarged view;

FIG. 2 illustrates a single pore of the flow diverting section when in the relaxed state;

FIG. 3 is an image showing a web formed by the coating material;

FIGS. 4A and 4B are SEM images of braided stents that were coated using the process disclosed herein;

FIG. 5 is a schematic view showing the flow loop test according to the present invention;

FIG. 6 is a schematic view of the plasma treatment head according to the present invention;

FIG. 7A-7F are schematic views of the coating on the sent according to the present invention.

### DETAILED DESCRIPTION OF THE INVENTION

Unless defined otherwise, all technical and scientific terms used herein have the same meanings as commonly understood by one of ordinary skill in the art. Although any methods and materials similar or equivalent to those described herein can be used in the practice or testing of the embodiments described herein, the preferred methods, devices, and materials are described herein. Further, although the present disclosure may refer to embodiments in which the medical device is a stent, the medical device can also be any implantable device, such as braided stent, coils, filters, scaffolds, expandable and balloon-expandable stents, and other devices.

In accordance with some embodiments disclosed herein, a medical device (e.g., stent) is provided that comprises an even coating that is inherently free of imperfections, such as lumps, fibers, webs, and/or other obstructions between any two elements of the device. Further, in some embodiments, such a device can be braided and/or have a flow diverting section that provides embolic properties so as to interfere with blood flow in (or into) the body space (e.g., an aneurysm) in (or across) which the device is deployed. The porosity and/or pore size of one or more sections of the device can be selected to interfere with blood flow to a degree sufficient to thrombose the aneurysm or other body space.

For example, some embodiments provide a device (e.g., stent) that can be configured to interfere with blood flow to generally reduce the exchange of blood between the parent vessel and an aneurysm, which can induce thrombosis of the aneurysm. A device (or a device component, such as a sidewall of a stent or a section of such a sidewall) that thus interferes with blood flow can be said to have a “flow diverting” property.

The medical device can comprise a tubular member having a sidewall and a plurality of pores in the sidewall that are sized to inhibit flow of blood through the sidewall into an aneurysm to a degree sufficient to lead to thrombosis and healing of the aneurysm when the tubular member is positioned in a blood vessel and adjacent to the aneurysm. The device can also have an anti-thrombogenic coating distributed over the tubular member such that the pores are free of webs formed by the coating.

In some embodiments, the tubular member comprising a plurality of filaments that are braided together to form pores therebetween. The tubular member can have a flow diverting section configured to span the neck of the aneurysm. The device can also have a coating distributed over the flow diverting section. The coating is distributed completely over the flow diverting section free of webs.

In some embodiments, the device can be an expandable stent made of two or more filaments. The filaments can be formed of known flexible materials including shape memory and superelastic materials, such as nitinol, platinum, Cobalt Chromium and stainless steel. In some embodiments, the filaments can be round or ovoid wire. Further, the filaments can be configured such that the device is expandable. In one embodiment, the stent can be fabricated from 32-micron DFT wire with a 40% Platinum core and be electro-polished prior to braiding. In other embodiments, one or more of the filaments can be formed of a biocompatible metal material or a biocompatible polymer. In another embodiment, one or more of the filaments can be composite wires which have a radiopaque core. The wires or filaments can be braided into a resulting lattice-like structure. In other embodiments, however, other methods of braiding can be followed, without departing from the scope of the disclosure. The device can exhibit a porosity configured to reduce haemodynamic flow into and/or induce thrombosis within, for example, an aneurysm, but simultaneously allow perfusion to an adjacent branch vessel whose ostium is crossed by a portion of the device. As will be appreciated, the porosity of the device can be adjusted by “packing” the device during deployment, as known in the art. The ends of the device can be cut to length and therefore remain free for radial expansion and contraction. The device can exhibit a high degree of flexibility due to the materials used, the density (i.e., the porosity) of the filaments, and the fact that the ends of the wires or filaments are not secured to each other on at least one of the ends of the stent.

Additionally, in some embodiments, a device (e.g., stent) can be provided with a porosity in the range of 5%-95% may be employed in the expanded braid.

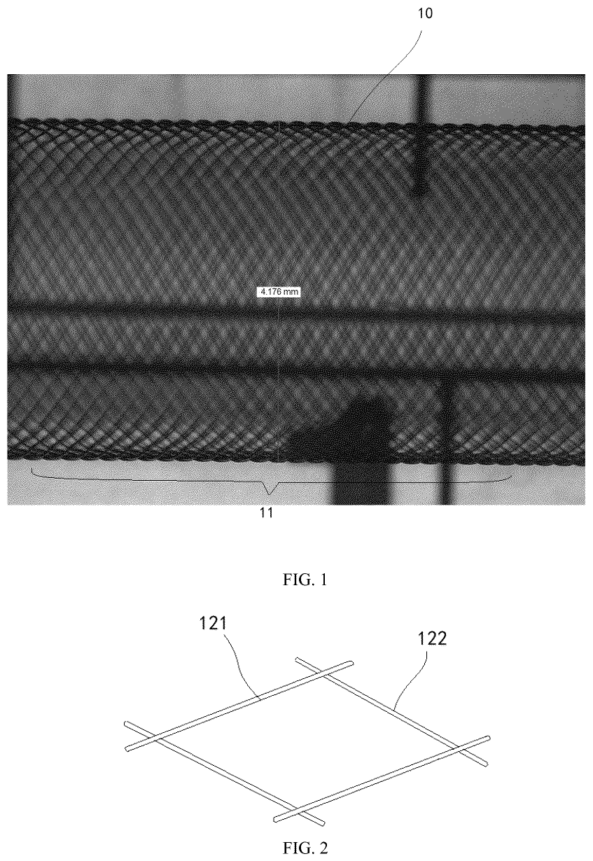

FIG. 1 illustrates a tubular, expandable device, shown as a stent 10, comprising a coating disposed along at least a portion thereof. The tubular stent 10 comprises an elongate hollow body which can be formed from a plurality of braided filaments. Some embodiments disclosed herein can comprise a coating along the entire length of the stent or merely along only a portion thereof. The stent 10 can comprise a flow diverting portion 11. The flow diverting portion 11 can comprise a plurality of pores that have a flow diverting pore size; instead of or in addition to this property, the flow diverting portion 11 can have a flow diverting porosity. The flow diverting portion 11 can comprise a portion of the stent 100, or the entire stent. The flow diverting pore size can be an average pore size within a relevant portion of the stent, e.g. within the flow diverting portion 11 or a portion thereof, or a “computed” pore size, one that is computed from measured or nominal basic stent parameters such as braid angle, number of filaments, filament size, filament diameter, stent diameter, longitudinal picks per inch, radial picks per inch, etc. Such a computed pore size can be considered to be one type of average pore size. The flow diverting pore size can be within a size range that interferes with or inhibits blood flow through the sidewall of the stent 100, for example, between the parent vessel and an aneurysm sufficient to induce or lead to thrombosis of the aneurysm. The coating can be disposed partially or entirely along the flow diverting portion 11, or along other portion of the stent 10.

In some embodiments, the pores of the flow diverting portion 11 can have an average pore size of less than 500 microns (inscribed diameter).

The average pore size of the pores in the flow diverting portion 11 can be the average size of the pores measured with or without coating material disposed thereon. Thus, the average pore size of the flow diverting portion of a bare stent can be within the flow diverting ranges. Further, the average pore size of the flow diverting portion of a coated stent can be within the flow diverting ranges. Furthermore, the flow diverting portion 11 can comprise pores having sizes above or below the range of the average pore size.

In some embodiment, the flow diverting portion 11 comprises a plurality of filaments that are braided together to form the tubular body of the stent 10. When the stent is in expanded or relaxed state, the filaments cross each other to form the pores of the stent 10.

FIG. 2 illustrates a single pore of the flow diverting section 11 when in the relaxed state. The pore is formed by a plurality of filaments. The angle between the two filaments can be any suitable angle in the prior art, e.g. the angle between the filaments 121, 122 can be from about 100° to about 170°.

Additionally, in order to maximize the pore size, in some embodiments, the filaments can form right-angled quadrilaterals, such as squares and/or rectangles. However, not every pore shape circumscribed by the filaments may be a right-angled quadrilateral, and some variation between pores in the same or different sections of a stent is possible.

In one preferred embodiment, the pore size and the expanded “at rest” diameter (i.e. the nominal diameter) of the stent would be set during the forming process by means of a heat treatment step. A resulting output of this heat setting step (in combination with the number of braid wires, the braiding angle, wire diameter, formed diameter, etc.) will be the pore size configuration (i.e. size and shape) that is inherent in that design. There is an optimal porosity and pore size from the perspective of the ability of the implanted braided structure to appropriately alter the blood flow into the aneurysm.

After the heat-setting step, the device can be cleaned and coated to impart desired surface characteristics to the device. Thereafter, the device can be coated using one of the methods disclosed herein.

The present invention discloses a plasma method that deposits the coating material on the medical device. The plasma method may be all known methods of plasma deposition that could be used to form a coating on the device.

The coating material can be one or more of a variety of anti-thrombogenic materials or platelet aggregation inhibitors, or anti-thrombogenic polymers or monomers. Suitable coating materials include 2-Methacryloyloxyethyl phosphorylcholine (MPC), PARYLENE C™, PARYLENE HT™, BAYMEDIX™, BIOCOAT™ hyaluronic acid, or polyethylene oxide. Other coating materials include heparin, heparin-like materials or derivatives, hirudin, H-Heparin, HSI-Heparin, albumin, phospholipids, streptokinase, tissue plasminogen activator (TPA), urokinase, hyaluronic acid, chitosan, methyl cellulose, poly (ethylene oxide), poly (vinyl pyrrolidone), endothelial cell growth factor, epithelial growth factor, osteoblast growth factor, fibroblast growth factor, platelet derived growth factor or angiogenic growth factor.

In some embodiments, a suitable form of MPC is prepared by mixing PC with methacrylate oils to get a stable MPC mixture, which is a preformed polymer. In other embodiments, a suitable form of MPC is 2-Methacryloyloxyethyl phosphorylcholine-poly (n-butyl methacrylate).

In some embodiment, the method involves preparing the solution of coating materials, such as preformed polymer; nebulizing the solution, for example with a gas, to form a liquid aerosol; polymerization is initiated in the plasma and the reacting fragments are deposited onto the surface of the medical device (e.g. stent) where they continue to react to form the complete coating. So the outcome is the polymer forming an insoluble network on the surface of the device, hence achieving the tenacity of the adhesion.

Plasma polymerization or glow discharge polymerization uses plasma sources to generate a gas discharge that provides energy to activate or fragment a gaseous or liquid monomer, often containing a vinyl group, in order to initiate polymerization. Polymers formed from this technique are generally highly branched and highly cross-linked, and adhere to solid surfaces well by means of chemical bonding. The biggest advantage of this process is that polymers can be directly attached to a desired surface while the chains are growing, which reduces the steps inherent with other coating processes such as grafting. This is very useful for pinhole-free coatings of 100 picometers to 1 micrometer thickness with solvent insoluble polymers.

Due to the amphiphilic nature of these polymers, the PC molecules have the potential of preferential orientation during the application process, so the hydrophobic portions interact with the device surface and the hydrophilic portions will present themselves at the surface upon hydration to provide the non-thrombogenic properties.

Plasma is an energetic gas containing ions, free radicals and other chemically reactive species. When these hit a metal surface, they can react with the oxide layer on the surface. This can clean the metal, disinfect it and also react with the metal surface leaving it temporarily covered in a layer of reactive species. Some of this involves breaking bonds on the surface of the metal. The more plasma power that is used on the surface, the more activated it becomes. A typical first step in the plasma deposition process is to use a high power plasma initially to clean the metallic surface and activate it. This is referred to as the pre-etching step. This leaves it covered in free radicals and oxidised molecules. The second step uses a low power plasma whilst introducing the liquid precursor. The coating process is mainly driven by free radicals. The free radicals preferentially attack the precursor at a particular chemical bond. In the case of the MPC coating, it's the carbon—carbon double bond that preferentially reacts. The precursor then starts to polymerise in the plasma. It travels through the plasma, further reacting, before hitting the surface. On the surface, the polymerisation reaction completes and this often involves direct reaction with the active species that we created on the metal oxide surface. The result is that polymerisation occurs in-situ on the surface resulting in a uniform coating that also binds directly to the active sites on the metal surface.

In another embodiment, large polymer biomolecules such as collagen and heparin can be deposited on the surface of the medical device. The method may involve preparing the solution of coating materials, such as preformed polymer; nebulizing the solution, for example with a gas, to form a liquid aerosol; polymerization is initiated in the plasma and the reacting fragments are deposited onto the surface of the medical device (e.g. stent) where they continue to react to form the complete coating.

In some embodiments, during the coating process the medical device (e.g. stent) is positioned within the plasma system, by means of holding the medical device using a mandrel or holder. The medical device is rotated during the coating process to coat the abluminal surface and the inner luminal surface. In other embodiments, the plasma system can envelop the entire medical device (e.g. stent) and hence the coating can be uniform and continuous without the need to rotate the device.

The amount of coating material applied to the medical device (e.g. stent) can vary depending upon numerous variables including, but not limited to the device itself and the coating material used. In an aspect, the amount of coating material can be sufficient to completely cover the device surface.

The application of the coating material using the plasma deposition process does not preclude the use of other surface treatment techniques as pre-treatment options. Such pre-treatment techniques may include, but are not limited to, electropolishing or passivation. Such pre-treatment techniques may have a beneficial effect on the adherence of the coating to the substrate but will not have a detrimental effect. In other embodiments, the device may be coated with multiple coating layers, for instance to produce a subbing layer, adding the oily monomer should provide a more flexible polymer film with hydrophobic properties to enhance physisorption.

The coated material(s) may be strongly bonded to the medical device, depending up on the operating parameters of the plasma (e.g., the level of plasma power employed), and the way in which the material(s) are exposed to the plasma. For example, in some embodiments, increasing the plasma power may increase the strength of cross-linking among coating material and/or may enhance the strength of bonding to the surface, which may result in the formation of covalent bonding between adjacent particles and/or covalent bonding of the coating material to the device.

Any suitable method can be used to nebulize the solution of coating material. This can include ultrasonic spray systems, rotary nozzles, electrospray devices, hydraulic nozzles, pneumatic spray or gas assisted spray systems. For gas assisted systems, any suitable gas can be used to nebulize the solution comprising the biomolecule. For example, the gas can be selected from the group consisting of nitrogen gas, helium gas, argon gas and mixtures thereof.

The plasma parameters (electrode design, frequency, voltage, gas composition, etc.) can be chosen to control the plasma process and ensure that the plasma operates in a non-thermal manner to produce a low-temperature plasma, which does not adversely affect temperature sensitive materials which are being deposited or the substrate material onto which they are being deposited.

Embodiments of the present invention employ non-thermal plasma devices where the plasma operates close to room temperature.

By directly introducing the coating material, as an aqueous spray into a low energy atmospheric pressure plasma, it has been found to produce a one-step route to the formation of stable, dry, adherent coatings.

A plasma device may comprise one or more electrodes and an ignition system operatively connected to the electrodes for providing a nonthermal equilibrium plasma. The plasma device may further comprise a gas supply inlet and a plasma chamber exposed to ambient pressure, wherein the non-thermal equilibrium plasma may be generated within the plasma chamber.

In at least one embodiment, the plasma device is a plasma coagulation device and the plasma produced by the device is introduced into a chamber alongside the coating material and/or at least one pharmaceutical. An end of the chamber may be open to atmosphere, and the medical device to be treated is placed adjacent to the exit of the chamber. This may result in plasma-treated materials depositing on the surface of the device as a coating.

The gas used to generate the plasma may comprise, e.g., helium or argon. For example, the device may comprise an argon plasma coagulator. In some embodiments, a helium plasma coagulator may be used, e.g., in place of the argon coagulator.

Plasmas can offer a number of advantages for coating deposition. The combination of reactive plasma and chemically-active monomers may produce a coating that is uniform, and/or well bonded to the medical device. Furthermore, curing of coating materials may occur in a manner that is almost instantaneous, which may offer processing advantages.

Some embodiments of the devices and methods disclosed herein can therefore provide a device, such as a stent or a braided stent, having a coating in which there is inherently no webbing between any two elements (such as filaments). There are several reasons why there is no webbing of material seen with this coating process. In one aspect, the coating process is without use of a solvent,-if the polymer is applied as a solution, it will have a certain surface tension and may inherently bridge across small gaps by capillary forces to form webs of polymer as the solvent evaporates. The utilization of a plasma phase material may avoid the above phenomenon associated with the application of a liquid phase coating through either a dipping or spraying application method.

In the device or stent according to the present invention, there is inherently no webbing because of the above plasma coating method. For example, in a braided stent, when the coating process is finished, there is no webbing formed between any wires of the stent at all. However, in the prior art, it cannot guarantee that there is no webbing formed between wires of the stent, or webbing will certainly be formed on the stent because of the coating method of prior art. It has to use additional steps to remove the webbing after coating, like blowing air to the coated stent. But when blowing air, the coating may have been deposited onto the surface of the stent and is hardly removed therefrom. Further, blowing air may cause drawbacks in the coating itself. Therefore, in the prior art, it cannot produce a stent or device with coating inherently no webbing.

The formation of webbing between any two elements in a densely braided structure is a common artefact of coating processes where there is significant “wetting” of the substrate during the application process. This typically occurs during dip or spray application processes where the coating solution is put down in such quantities that a post-processing step (for example the use of an “air-knife” to blow off excessive coating solution) is necessary to reduce the coating thickness to acceptable levels. Even with such post-processing steps, the surface tension of the coating solution makes it possible/likely that webbing can remain between neighboring elements in a braided structure. Typically, a curing step is required after the application of the coating and this “freezes” any webs into position.

On the other hand, the use of plasma deposition is a fundamentally different way of applying a coating material. The material to be deposited is placed into solution using an alcohol as the solvent. Pre-etching of the substrate together with an energized cold plasma corona (into which the coating material is nebulized) results in the coating layer being built up from zero thickness to the intended thickness. A covalent bond forms between the substrate and the coating material instantaneously upon delivery of the coating material whilst the alcohol solvent flashes off leaving only the solute coating material behind. Curing of the coating material happens automatically since adjacent molecular chains cross-link spontaneously, effectively eliminating surface tension as a mechanism that could enable the gathering of the wet coating solution at a junction between adjacent wires. Hence webbing of the coating is not seen as a feature that can occur as part of a plasma deposition coating process.

That is why the devices and methods according to present invention can therefore provide a device, such as a stent or a braided stent, having a coating in which there is inherently no webbing between any two elements (such as filaments).

In some embodiments that also has a flow diverting pore size and/or a flow diverting porosity, the flow diverting aspect of the stent is exhibited throughout the entire stent, or in just a section of the stent.

In some embodiments, the medical device, such as a stent, may be coated along the entire length. In other embodiment, the medical device, such as a stent, may be coated along the length of flow diverting portion.

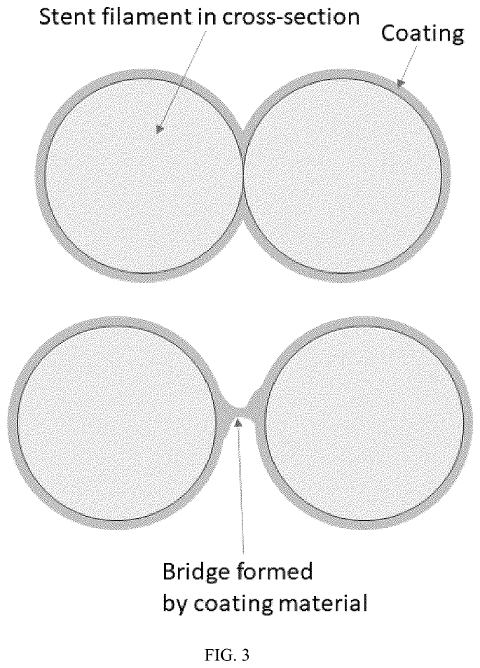

FIG. 3 is an image showing a web formed by the coating material. In FIG. 3, the upper portion shows two elements (filaments) of the device are in contact with one another and the coating is continuous from to the other but does not form a bridge as the elements are touching, while the lower portion shows two elements of the device are not in contact with one another but the coating links one filament to the other through a bridge. This bridge part constitutes the webbing between the two elements.

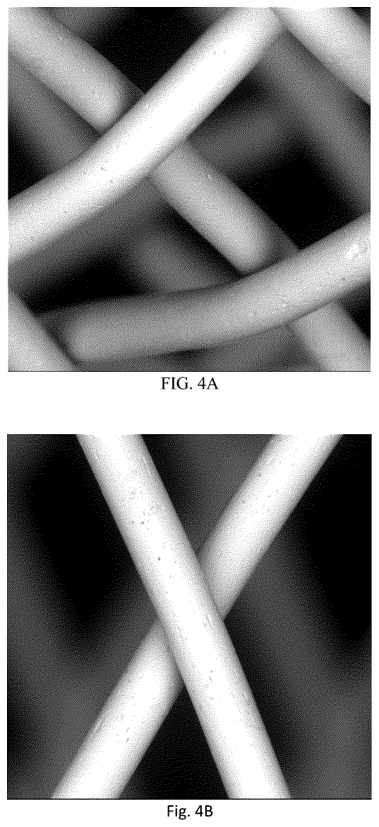

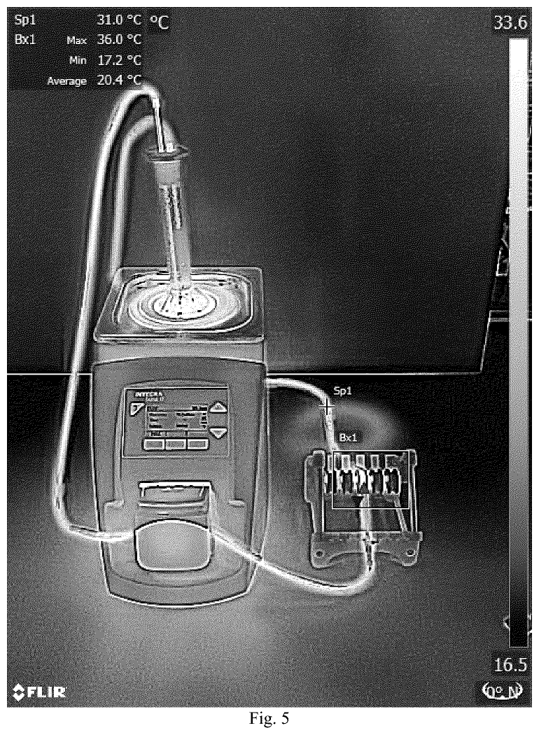

The coated stent can be observed in SEM (scanning electron microscope) imaging. FIGS. 4A and 4B are SEM images of braided stents that were coated using the process disclosed herein.

According to one embodiment of the present invention, in the medical device for treating an aneurysm, an antithrombogenic material is coated over the flow diverting portion according to the above method, and a coating of the antithrombogenic material formed thereon has a thickness equal to or greater than 10 nm. Preferably, the coating of the antithrombogenic material has a thickness of 10-200 nm. More preferably, the coating of the antithrombogenic material has a thickness of 10-60 nm, 10-100 nm, 20-30 nm, 30-40 nm, 30-50 nm, 40-60 nm, 60-80 nm, 80-100 nm, 100-120 nm, 100-150 nm, 120-130 nm, 130-150 nm, 150-180 nm, 150-200 nm, 160-180 nm, or 180-200 nm.

In the present invention, the coating is deposited on the medical device by the plasma method disclosed above. With such a method, the antithrombogenic material is adhered to the surface of the medical device by means of chemical bonding, and thus the coating formed therefrom has a very good adhesion on the medical device.

As a result, the coating may have a thickness of 10-200 nm or even more, as mentioned above. Compared with the prior art, in which such a coating usually has a thickness of 3 nm or even less, the coating according to the present invention may have an overwhelming advantage in anti-thrombotic performance.

Further, because of the good adhesion, the coating according to the present invention may have a good firmness and durability, and is hardly to peel off from the medical device during use, which is extremely good for such medical devices used in human body, especially in the blood vessels.

FIG. 5 is a schematic view showing the flow loop test according to the present invention, the result of which indicates the good firmness and durability of the coating according to the present invention.

As shown in FIG. 5, the general idea is to hold a stent with the coating according to the present invention in a challenge position and pump saline around it for 28 days. The thermal image is to ensure the stent is still seeing 37° C. saline and there is no drop of in temperature in the loop.

There are 4 stents and 6 analysis points: Week 2—one point, Week 3—one point, Week 4—2×stents and 2×analysis points on each.

On some of the lower magnification images, a lot of crystal deposits are seen but this is just salt residue from the saline. The real information is in the high-resolution cross-sectional images. They show no change from 2 weeks to 4 weeks. In fact, they look identical to the initial samples that were never subjected to the saline loop test. So it is now logical to assume that the coating is both adherent and durable when subjected to aggressive flow for up to a 4 week implantation period.

According to the above embodiment of the present invention, the antithrombogenic material is coated onto the tubular member of the medical device through a plasma deposition method such that there is no webbing of the antithrombogenic material between any two elements.

According to the above embodiment of the present invention, the coating of the antithrombogenic material on the tubular member has a super-smooth surface. Preferably, the surface of the coating of the antithrombogenic material has an average roughness of about 0.02-0.2 μm, for example 0.1 μm and/or a Mean Roughness depth of about 0.2-2 μm, for example 0.5 μm. The coating has such a super-smooth surface and the above roughness due to the plasma depositing method for depositing the antithrombogenic material on the tubular member of the medical device.

In one embodiment of the present invention, on the braid wires for the medical device, there is provided with Blue Oxide surface. The Blue Oxide surface finish purports to have superior smoothness because it is subjected to an electro-polishing step prior to being wound into the braided component. The blue hue on the material comes from the fact that the heat treatment step is the final processing step that the component sees and the resulting oxidation layer persists and exhibits this colouration. The Blue Oxide surface on the wire has an average roughness of about 0.02-0.2 μm, for example 0.1 μm and/or a Mean Roughness depth of about 0.2-2 μm, for example 0.5 μm.

When the coating is deposited on Blue Oxide surface of the wire by the plasma method disclosed above, the surface of the coating of the antithrombogenic material also has an average roughness of about 0.02-0.2 μm, for example 0.1 μm and/or a Mean Roughness depth of about 0.2-2 μm, for example 0.5 μm.

It is evident that the Blue oxide surface finish has a smoothness that is 10 times better than that of the standard surface finish. On the basis of the measurements obtained, it is clear that there are significant benefits in terms of surface smoothness when the wire is subjected to electro-polishing prior to being braided. The coating onto the Blue oxide via the plasma depositing method has almost the same smoothness as the Blue oxide, and can exhibit super smoothness.

As mentioned elsewhere herein, the present disclosure also includes methods of treating a vascular condition, such as an aneurysm or intracranial aneurysm, with any of the embodiments of the coated stents disclosed herein. The coated, low-thrombogenicity stent could be deployed across the neck of an aneurysm and its flow-diverting properties employed to reduce blood flow between the aneurysm and the parent vessel, cause the blood inside the aneurysm to thrombose and lead to healing of the aneurysm.

In order to implant any of the coated stents disclosed herein, the stent can be mounted in a delivery system. Generally, the delivery system can include an elongated core-wire assembly that supports or contains the stent, and both components can be slidably received in a lumen of a microcatheter or other elongated sheath for delivery to any region to which the distal opening of the microcatheter can be advanced. The core-wire assembly is employed to advance the stent through the microcatheter and out the distal end of the microcatheter so that the stent is allowed to self-expand into place in the blood vessel, across an aneurysm or other treatment location.

A treatment procedure can begin with obtaining percutaneous access to the patient's arterial system, typically via a major blood vessel in a leg or arm. A guidewire can be placed through the percutaneous access point and advanced to the treatment location, which can be in an intracranial artery. The microcatheter is then advanced over the guidewire to the treatment location and situated so that a distal open end of the guidewire is adjacent to the treatment location. The guidewire can then be withdrawn from the microcatheter and the core-wire assembly, together with the stent mounted thereon or supported thereby, can be advanced through the microcatheter and out the distal end thereof. The stent can then self-expand into apposition with the inner wall of the blood vessel. Where an aneurysm is being treated, the stent is placed across the neck of the aneurysm so that a sidewall of the stent (e.g. a section of the braided tube) separates the interior of the aneurysm from the lumen of the parent artery. Once the stent has been placed, the core-wire assembly and microcatheter are removed from the patient. The stent sidewall can now perform a flow-diverting function on the aneurysm, thrombosing the blood in the aneurysm and leading to healing of the aneurysm.

### Plasma Treatment Head

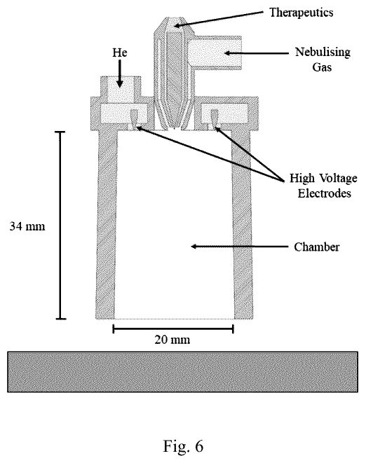

In some embodiments, a plasma treatment head shown in FIG. 6 is used for performing the plasma treatment. The plasma treatment head includes the following main components:
Supply of therapeutic/material to be deposited;
Supply of inert gas (in some embodiments Helium);
High Voltage Electrodes; and
Chamber.

Helium gas flowing adjacent to the high voltage electrodes produces an energized cold temperature plasma into which the coating material is injected through a nebulizing head. The chamber contains the coating-material-and-energized-gas combination so that it remains local to the component to be treated, such a stent to be coated according to the present invention. The energy in the plasma causes active sites in the molecular chains of the coating material to form.

In some preferable embodiments, a pre-coating plasma etching of the substrate (carried out under vacuum conditions in a separate plasma etching machine) may be provided, which causes active sites to form on the surface of the substrate. The part to be coated is placed into the chamber of the pre-etching machine, such as Diener ZEPTO or other existing machines on the market, and a vacuum is pulled. The power of the system to generate a plasma is variable from 50 to 400 Watts. The time for the pre-etching step is 0.1-20 minutes. Once the pre-etching is complete, the chamber is vented and the parts are removed. They are immediately placed into the plasma deposition machine for coating of the PC material although this is not strictly necessary since the activation of the surface will persist for a number of hours after the pre-etching step.

These active sites, together with those formed on the molecular chains of the coating material, result in a covalent bond forming between the coating material and the substrate. Crosslinking between molecular chains of the coating material will occur during this process but the objective is to minimize these as much as possible in order to ensure that the PC chains can reorientate themselves when in contact with an aqueous environment.

In some embodiments, a layer of blue oxide is provided over the flow diverting portion, and the antithrombogenic material is coated over the layer of blue oxide.

In some embodiments, the coating of the antithrombogenic material on the tubular member has a super-smooth surface. The surface of the coating of the antithrombogenic material has an average roughness of about 0.02-0.2 μm, for example 0.1 μm and/or a Mean Roughness depth of about 0.2-2 μm, for example 0.5 μm.

In some embodiments, the coating of the antithrombogenic material has a thickness of 10-200 nm, and preferably 30-50 nm.

### Outline of Process and Parameters

Several parameters can be varied in order to change the ultimate configuration of the applied coating material on the surface of the substrate or the stent. These parameters broadly fall under 4 different categories:
1) Plasma energy parameters;
2) Coating solution and concentration parameters;
3) Pre-etching parameters; and
4) In-process parameters.

### Plasma Energy Parameters

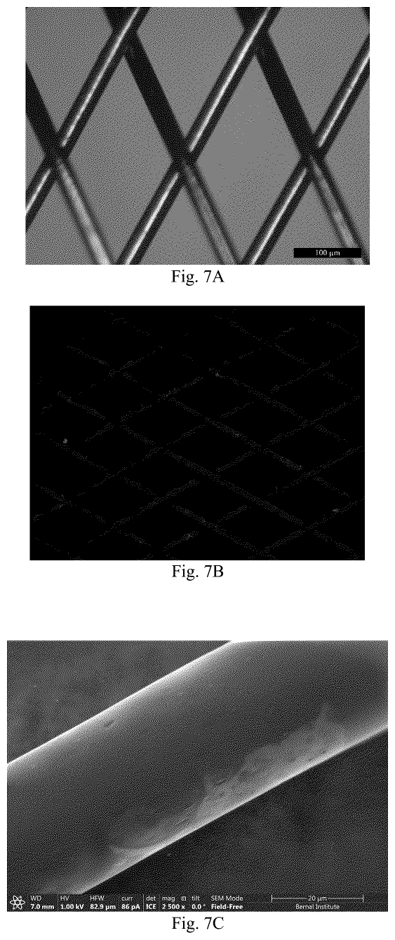

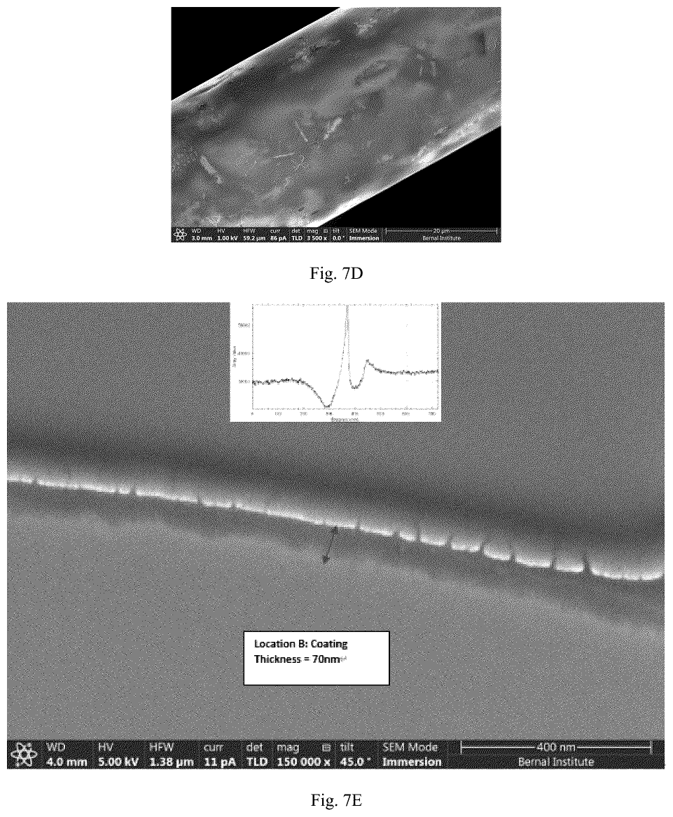

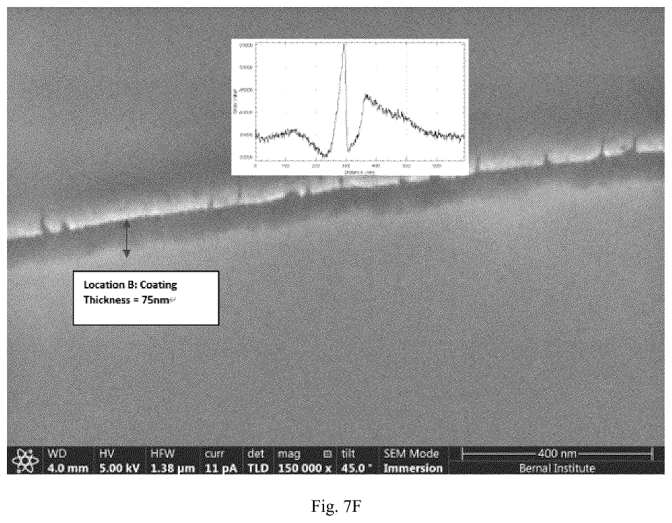

A prescribed sinusoidal voltage and current is applied across the two electrodes in the plasma treatment head. The magnitude and temporal pattern of these two parameters determine the power applied to the plasma field. Typically, the voltage and currents are applied for a short pulse period (referred to as the “pulse on time”) followed by a period when the voltage and currents are off (referred to as the “pulse off time”), see FIG. 7. Both times can be varied independently so that the overall power applied to the plasma field can also be varied.

The overall power delivered to the high voltage electrodes (and therefore applied to the plasma field) is given by:

$$Power = \frac{1}{M} {\sum }_{k = 0}^{M} {V}_{k} {I}_{k}$$

V is the voltage applied and I is the current applied, M is the total number of the pulse applied to the plasma treatment head.

For instance, a voltage is 1-200V, a pulse on time is 1-200 ms, a pulse off time is 1-200 ms, a power output may preferably be in the range of 1-20 W.

### Coating Solution and Concentration Parameters

The coating solution is produced by dissolving a quantity of phosphorylcholine pre-formed co-polymer into an alcohol and diluting the resulting solution using de-ionized water. Two different alcohol types have been assessed as well as various water: alcohol concentrations. Finally, the aging time of the final solution can be varied as a process parameter.

In some embodiments of the invention, the following solution and concentration parameters can be considered for the coating process:
1) water: alcohol concentration of 90:10-10:90;
2) Choice of alcohol: ethanol vs. methanol
3) Aging time of solution prior to coating process.

### Pre-Etching Parameters

The degree to which the surface of the substrate is etched prior to the coating process can also be varied. This has the effect or varying the level of adhesion between the coating material and the substrate.

In some embodiments, the power of the system to generate a plasma is variable from 50 to 400 Watts. The time for the pre-etching step is 0.1-20 minutes.

### In-Process Parameters

The way in which the part to be treat, or the stent, is presented to the plasma deposition head can be varied. The head passes over the stent several times and at a set speed. A typical configuration of the head settings in the embodiment are as follows:
1) Head speed is 300-2000 mm/min;
2) Number of passes over the part is 1-20.

In some embodiments, the above discussed parameters may be set as follows:
Water: ethanol mix is 90:10-10:90, for example, 80:20, 70:30, 60:40, 40:60, 30:70, 20:80, 90:10;
Concentration of PC in solution is 0.5-20 mg/ml, for example, 0.7 mg/ml, 1 mg/ml, 2 mg/ml, 3 mg/ml, 5 mg/ml, 7 mg/ml, 10 mg/ml, 12 mg/ml, 13 mg/ml, 17 mg/ml, 20 mg/ml;
Power measure at plasma head is 1-20 W, for example, 1 W, 2 W, 4 W, 5 W, 6 W, 8 W, 10 W, 14 w, 18 w, 20 W;
Pre-etch setting time is 0.1-20 minutes, for example, 0.1 mins, 0.5 mins, 0.8 mins, 1 mins, 4 mins, 6 mins, 10 mins, 15 mins, 18 mins, 20 mins;
Head speed is 300-2000 mm/minute, for example, 300 mm/min, 500 mm/min, 700 mm/min, 900 mm/min, 1200 mm/min, 1500 mm/min, 1800 mm/min, 2000 mm/min; and
Number of passes over the part is 1-20, for example, for example, 1, 3, 6, 7, 8, 10, 15,17, 20.

A variety of options for the above parameters can be adjusted according to the target coating thickness to obtain consistent results. A sent is plasma coated based on the above process. The parameters of its plasma treatment are selected in the above ranges.

For the above stent, FIG. 7A is its visual microscopy image.

FIG. 7B is its fluorescence image. A method for visualizing the presence of Phosphorylcholine coating is to stain the material using Rhodamine dye and imaging the structure under fluorescent light.

FIGS. 7C and 7D are its SEM images.

FIGS. 7E and 7F are its coating cross sections.

The locations where the thickness measurements were taken were completely at random so it is reasonable to assume that the observed range is representative of the coating over the entire structure. To further bolster this viewpoint, comparisons were taken using imaging from SEM, visual microscopy and fluorescence. The visual microscopy shows a consistent blue-hue colouration consistently throughout the structure—this is in contrast to other Trial samples where the blue-hue also contains regions of multi-colouration. When this stent is examined using fluorescence, there is a pretty consistent red colouration (low brightness) with little if any high-brightness specs. Finally, SEM imaging of this stent shows a reasonably uniform surface topology without any of the circular patches, clumps or flake-like features that are present in practically other samples.

The devices and methods discussed herein are not limited to the coating of stents, but may include any number of other implantable devices. Treatment sites may include blood vessels and areas or regions of the body such as organ bodies.

The foregoing description, for purpose of explanation, has been described with reference to specific embodiments according to the present invention. However, the illustrative descriptions above are not intended to be exhaustive or to limit the invention to the precise forms disclosed.

1. A medical device, comprising:
an expandable tubular member comprising a plurality of elements forming a sidewall, wherein each of the elements crosses one or more other elements and a plurality of pores are formed among the elements in the sidewall;
wherein an antithrombogenic material is coated over the tubular member such that there is inherently no webbing of the antithrombogenic material between any two elements.

2. The medical device according to claim 1, wherein:
the tubular member having at least a flow diverting portion, the flow diverting portion comprising a plurality of elements forming a sidewall of the tubular member, wherein each of the elements crosses one or more other elements and a plurality of pores are formed among the elements in the sidewall;
wherein the pores are sized to interfere with blood flow to a degree sufficient to thrombose the aneurysm when the tubular member is expanded;
wherein an antithrombogenic material is coated over the flow diverting portion such that there is inherently no webbing of the antithrombogenic material between any two elements.

3. The medical device according to claim 1, wherein the medical device is for treating an aneurysm.

4. The medical device according to claim 1, wherein the antithrombogenic material is coated onto the tubular member through a plasma deposition method.

5. The medical device according to claim 4, wherein the plasma deposition method is a non-thermal plasma deposition method.

6. The medical device according to claim 1, wherein the medical device is a stent, each of the elements is a filament, and the stents are formed by braiding the filaments.

7. The medical device according to claim 1, wherein the tubular member is coated with the antithrombogenic material in its entirety.

8. The medical device according to claim 1, wherein the tubular member is coated with the antithrombogenic material on its inner luminal surface.

9. The medical device according to claim 1, wherein a layer of blue oxide is provided over the tubular member, and the antithrombogenic material is coated over the layer of blue oxide.

10. The medical device according to claim 1, wherein the coating of the antithrombogenic material on the tubular member has a super-smooth surface.

11. The medical device according to claim 10, wherein the surface of the coating of the antithrombogenic material has an average roughness of about 0.02-0.2 μm, and particularly 0.1 μm, and/or a Mean Roughness depth of about 0.2-2 μm, and particularly 0.5 μm.

12. The medical device according to claim 1, wherein the antithrombogenic material is adhered to the surface of the medical device by means of chemical bonding.

13. The medical device according to claim 12, wherein a covalent bonding is formed between adjacent particles of the antithrombogenic material and/or a covalent bonding is formed between the antithrombogenic material and the surface of the medical device or the layer of blue oxide during coating; and wherein the antithrombogenic material comprises an antithrombogenic polymer.

14. (canceled)

15. A method for coating a medical device, which has a plurality of elements crossing with each other, comprising:
preparing a solution of antithrombogenic material;
nebulizing the solution to form a liquid aerosol;
generating plasma with a plasma source;
introducing the liquid aerosol into the plasma; and
depositing the medical device with a coating of the antithrombogenic material by exposing the medical device to the liquid aerosol and the plasma, so that there is inherently no webbing of the antithrombogenic material between any two elements of the medical device.

16. The method according to claim 15, wherein the medical device is for treating an aneurysm.

17. The method according to claim 15, wherein the plasma operates in a non-thermal manner.

18. The method according to claim 15, wherein before exposing the medical device to the liquid aerosol and the plasma, a pre-coating plasma etching is performed on the medical device.

19. The method according to claim 15, wherein the antithrombogenic material is adhered to the surface of the medical device by means of chemical bonding, and particularly by covalent bond.

20. The method according to claim 15, wherein the medical device is a stent, each of the elements is a filament, and the stent is formed by braiding the filaments.

21. The method according to claim 15, wherein the antithrombogenic material comprises an antithrombogenic polymer.

22-36. (canceled)
